(19) United States
(12) Patent Application Publication
(10) Pub. No.: US 2011/0196509 A1
(43) Pub. Date: Aug. 11, 2011

(54) HYDRAULIC APPARATUS WITH DIRECT TORQUE CONTROL

(75) Inventors: John F. Jansen, Knoxville, TN (US); Randall Frank Lind, Loudon, TN (US); Lonnie J. Love, Knoxville, TN (US)

(73) Assignee: UT-Battelle, LLC, Oak Ridge, TN (US)

(21) Appl. No.: 12/970,185

(22) Filed: Dec. 16, 2010

Related U.S. Application Data
(63) Continuation-in-part of application No. 12/394,276, filed on Feb. 27, 2009.

(30) Foreign Application Priority Data
Feb. 24, 2010 (US) PCT/US2010/025199

(51) Int. Cl. A61F 2/74; F15B 13/06; F15B 15/20

(52) U.S. Cl. 623/26; 91/499; 91/471

(57) ABSTRACT

A hydraulic apparatus has a cam body rotatable about an axis and with at least two mirror image involute cam surfaces on opposing sides of the axis. Hydraulic actuators on opposing sides of the axis have a linearly extendable ram and a hydraulic cylinder. A fluid supply line is provided for delivering a pressurized fluid equally to each of the cylinders. A pressure relief or flow control valve is associated with each of the hydraulic cylinders and is operable to selectively assume a closed position to retain fluid or assume an open position to release fluid from the hydraulic cylinders. A control can operate the pressure relief valves between at least the closed and open positions, to move the rams out of and in to the cylinders to rotate the cam body about the axis with controlled torque. A robotic, prosthetic or orthotic and a method for applying torque to a body are also disclosed.

### CROSS-REFERENCE TO RELATED APPLICATIONS

This application is a continuation-in-part of U.S. patent application Ser. No. 12/394,276, filed Feb. 27, 2009, and also claims priority to International Application No. PCT/US2010/025199, filed Feb. 24, 2010, the entireties of which are incorporated by reference herein.

### STATEMENT REGARDING FEDERALLY SPONSORED RESEARCH OR DEVELOPMENT

This invention was made with government support under DE-AC05-00OR22725 awarded by The United States Department of Energy. The government has certain rights in the invention.

### FIELD OF THE INVENTION

This invention relates to actuators, and particularly to hydraulic actuators useful for haptic devices.

### BACKGROUND OF THE INVENTION

Actuators convert energy (electrical, mechanical, chemical, thermal) to mechanical motion. Examples include solenoids, electric motors, hydraulic and pneumatic pistons, piezoelectrics, shape memory alloys and electroactive polymers. The predominant form of actuation in industry is shared by electric motors and hydraulic and pneumatic pistons. Hydraulic pistons dominate the heavy industries (construction, aerospace, manufacturing) where high forces and high power density are a necessity.

Electric motors have two primary means for converting electrical to mechanical energy. Constant current controls torque whereas providing constant voltage controls speed. Hydraulic actuators have one primary control mode: constant flow controls speed. Conventional hydraulic joints are motion controlled (position or velocity controlled). There is no direct way of directly controlling torque to a conventional hydraulic joint.

Existing haptic devices use torque controlled electric motors. These devices can be heavy and can generate a significant amount of heat. Hydraulic actuators have not frequently been used in haptic and other prosthetic devices due to weight, control issues and other concerns. Also, hydraulic actuators can lock up when they malfunction, which is not desirable in a prosthetic or haptic device.

Devices with hydraulic involute cam actuators have been described in WO 2010/099175, the disclosure of which is incorporated by reference. Such devices provide for the efficient transmission of the linear motion of hydraulic actuators into torque.

### SUMMARY OF THE INVENTION

A hydraulic apparatus with direct torque control includes a cam body, the cam body being rotatable about an axis and having at least two mirror image involute cam surfaces on opposing sides of the axis. At least two hydraulic actuators are provided on opposing sides of the axis. Each of the actuators has a linearly extendable ram and a hydraulic cylinder. The ram has a piston positioned in the hydraulic cylinder and each ram is extendable to contact the involute cam surface on the respective opposing side of the axis. A fluid supply line is provided for delivering a pressurized fluid to each of the cylinders. An open fluid connection is provided between the hydraulic cylinders. A pressure relief or flow control valve is associated with each of the hydraulic cylinders. The pressure relief or flow control valves are operable to selectively assume a closed position to retain fluid in the hydraulic cylinders or assume an open position to release fluid from the hydraulic cylinders. A control operates the pressure relief or flow control valves between at least the closed and open positions, to extend and retract the rams out of and in to the cylinders to rotate the cam body about the axis with controlled torque.

A fixed area fluid supply orifice can be associated with each hydraulic cylinder. The supply orifices have an equal area and are connected to a common supply of pressurized fluid. The cylinders can be provided in a cylinder housing, and the cam body can be pivotally connected to the cylinder housing through the axis.

A tool can be connected to the can body, whereby movement of the cam body will cause movement of the tool. The tool can be a robotic, prosthetic or orthotic device. The robotic, prosthetic or orthotic device can be selected from the group consisting of a hand, a shoulder, an arm, an elbow, a finger, a foot, an ankle, a knee, a hip, and a leg.

A barrel can and cam follower can be provided to impart rotation about an axis orthogonal to the axis of rotation of the cam body. The cam body can include two coincident pairs of involute cam surfaces. A second hydraulic apparatus can be provided and connected in series to the other hydraulic apparatus. The valves can be any suitable valve, such as poppet valves.

A robotic, prosthetic or orthotic device includes a hydraulic device. The hydraulic device has a cam body, the cam body being rotatable about an axis and having at least two mirror image involute cam surfaces on opposing sides of the axis. At least two hydraulic actuators are provided on opposing sides of the axis. Each of the actuators has a linearly extendable ram and a hydraulic cylinder. The ram has a piston positioned in the hydraulic cylinder and is extendable to contact the involute cam surface on the respective opposing side of the axis. A fluid supply line is provided for delivering a pressurized fluid to each of the cylinders. A pressure relief valve is associated with each of the hydraulic cylinders, the pressure relief valves being operable to selectively assume a closed position to retain fluid in the hydraulic cylinders or assume an open position to release fluid from the hydraulic cylinders. A control operates the pressure relief valves between at least the closed and open positions, to extend and retract the rams out of and in to the cylinders to rotate the earn body about the axis with controlled torque.

A robotic, prosthetic or orthotic tool can be connected to the cam body. The robotic, prosthetic or orthotic tool is selected from the group consisting of a hand, a shoulder, an arm, an elbow, a finger, a foot, an ankle, a knee, a hip, and a leg. The robotic, prosthetic or orthotic device can include first and second hydraulic devices connected in series. The first hydraulic device can be connected to a robotic, prosthetic or orthotic hand. The robotic, prosthetic or orthotic hand can have a plurality of pairs of first and second hydraulic device digits, wherein the first hydraulic devices are connected to the robotic, prosthetic or orthotic hand, and another pair of first and second hydraulic devices connected to the hand as an opposable thumb.

A method of applying torque to a body, includes the steps of:

providing a hydraulic device, said hydraulic device comprising a cam body, the cam body being rotatable about an axis and having at least two mirror image involute cam surfaces on opposing sides of the axis;

at least two hydraulic actuators on opposing sides of the axis, each of the actuators having a linearly extendable ram and a hydraulic cylinder, the ram comprising a piston positioned in the hydraulic cylinder and the ram being extendable to contact the involute cam surface on the respective opposing side of the axis;

a fluid supply line for delivering a pressurized fluid to each of the cylinders;

an open fluid connection between the hydraulic cylinders;

a pressure relief or flow control valve associated with each of the hydraulic cylinders, the pressure relief or flow control valves being operable to selectively assume a closed position to retain fluid in the hydraulic cylinders or assume an open position to release fluid from the hydraulic cylinders; and

a control for operating the pressure relief or flow control valves between at least the closed and open positions, to extend and retract the rams out of and in to the cylinders to rotate the cam body about the axis with controlled torque;

applying fluid pressure equally to the hydraulic cylinders to provide no torque on the cam body about the axis;

selectively releasing pressure in at least one of the hydraulic cylinders by operating a respective pressure relief valve, to create a hydraulic pressure differential between the hydraulic cylinders, the pressure differential creating a torque on the cam body about the axis and a net force on the respective ram of the reduced pressure hydraulic cylinder, causing the ram to retract into the respective hydraulic cylinder.

### BRIEF DESCRIPTION OF THE DRAWINGS

A fuller understanding of the present invention and the features and benefits thereof will be obtained upon review of the following detailed description together with the accompanying drawings, in which:

FIG. 1 is a schematic view of a hydraulic apparatus according to the invention.

FIG. 2 is a cross section of a hydraulic apparatus according to the invention.

FIG. 3 is a cross section of a hydraulic apparatus with an involute cam and a barrel cam.

FIG. 4 is a perspective view of a barrel cam.

FIG. 5 is a perspective view of a hydraulic apparatus having two coincident involute cams.

FIG. 6 is a perspective view of the apparatus of FIG. 5 with outer housings removed to reveal internal features.

FIG. 7 is a perspective view of a cam body with two coincident involute cams.

FIG. 8 is a perspective view of a wearable mesofluidic glove.

FIG. 9 is a perspective view of a wearable finger mechanism.

FIG. 10 is a cross section of the wearable finger mechanism.

### DETAILED DESCRIPTION OF THE INVENTION

The subject of this invention is a hydraulic apparatus that enables direct control of torque. The invention provides direct torque control, using fluid power, of a compact mechanical apparatus such as a prosthetic joint. The joint can provide high torques with less volume and weight than electric motors, and remains very cool, which is important for wearable devices. This invention provides a direct means to provide torque proportional to an input electrical signal. This characteristic is critical for wearable force controlled devices. The joint is free to move when there is no electrical power. Therefore it is fail safe. Likewise, resistance torque increases with increasing electrical command.

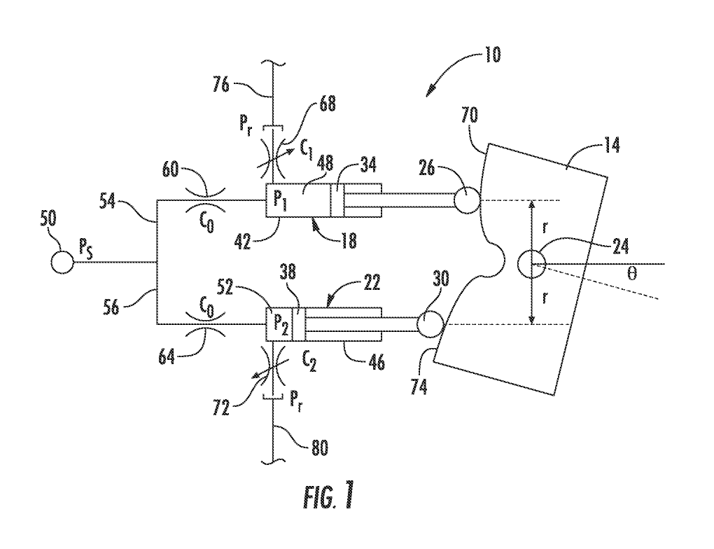

As shown in FIG. 1, the joint assembly 10 has an involute cam body 14 and a pair of actuators 18 and 22. The cam body 14 is rotatable about an axis 24. The actuators 18 and 22 have rams 26 and 30, which are connected to pistons 34, 38 positioned within cylinders 42 and 46 providing chambers 48, 52. Pressure Pₛ from a supply 50 is directed through supply line 54, 56 and fixed orifices 60, 64 having an area of C_{O}. Pressure relief or flow control valves 68, 72 are associated with the cylinders 42 and 46 to return lines 76, 80. The pressure relief or flow control valves can control the flow from the piston chambers 48 and 52 through conventional variable orifices or full on-full off digital flow control valves. The supply pressure to the cylinders can be the same or different, although for ease of control the supply pressure is preferably the same. Pressure in the cylinders P_{C1} and P_{C2} can be the same or different, depending on the position of the pressure relief or flow control valves 68, 72. The return pressure through the return lines 76, 80 is Pᵣ₁ and Pᵣ₂.

The cam body 14 has mirror image involute surfaces 70, 74 on opposing sides of the axis 24. The actuators 18 and 22 are also aligned on respective opposing sides of the axis 24, such that extending of the rams 26, 30 out of the cylinders 42, 46 will cause the rams to contact the respective involute surfaces 70, 74.

Equation (1) is the general curve that defines the involute cam surface:

x=r(cos(θ)+θ sin(θ))

y=r(sin(θ)−θ cos(θ)) (1)

There are several advantages to this cam profile. First, the transmission ratio, the conversion of actuator force to cam body torque, is constant independent of the cam orientation θ. Traditional slider crank mechanisms experience a 2:1 variation in transmission ratios through 120 degrees of motion. Therefore, the actuators must be oversized (by a factor of two) to ensure peak torque through the full range of motion. This is not the case for the involute cam actuator. The pressure angle between the rams 26, 30 and the involute cam surfaces 70, 74 is 0° through the full range of motion of the cam. This ensures very low friction, no side loading on the rams and efficient transmission of actuator forces to cam body torque. Torque is controlled by the antagonistic actuators 18, 22, which reduces backlash during operation. Also, because the actuators 18, 22 are stationary, fluid can be routed to the actuators through fixed supply lines rather than flexible hoses. This increases reliability and decreases the chance of leakage.

Equation (2) expresses the torque as a function of the actuator pressures (P₁ and P₂) where Aₚ, is the piston cross sectional area:

τ=rAₚ(P₁−P₂) (2)

Therefore, providing direct control of torque requires direct control of pressure. The invention can use a fixed orifice between the supply pressure Pₛ and chambers 48, 52, and pressure relief valves 68, 72 between the chambers 48, 52 and the return pressure Pᵣ₁ and Pᵣ₂. The fixed orifice area (Cₒ) can be approximately ½ the cross sectional area of the orifices C₁, C₂ of the pressure relief valves 68, 72. When both of the pressure relief valves 68, 72 are closed, both chamber pressures are P. Since the actuators 18, 20 have the same cross sectional area and the transmission ratio (r, due to the involute cam) is independent of cam body angle, the net torque is zero. If one of the valves is opened, the pressure in the respective chamber will drop producing a net torque proportional to the valve opening—torque will be proportional to the valve command.

For the two chambers, the pressures are:

$$\frac{d {P}_{1}}{d t} = \frac{β}{{V}_{1}} \{ {C}_{o} {C}_{d} \sqrt{\frac{2}{ρ} ( {P}_{s} - {P}_{1} )} - {C}_{1} {C}_{d} \sqrt{\frac{2}{ρ} {P}_{1}} - {A}_{p} r \ddot{θ} \} \\ \frac{d {P}_{2 }}{d t} = \frac{B}{{V}_{2}} \{ {C}_{o} {C}_{d} \sqrt{\frac{2}{ρ} ( {P}_{s} - {P}_{2} )} - {C}_{2} {C}_{d} \sqrt{\frac{2}{ρ} {P}_{2}} + {A}_{p} r \dot{θ} \} \qquad ( 3 )$$

where C_{d} is the discharge coefficient, and V₁ and V₂ are the chamber volumes. It is clear from the equations that opening either of the pressure relief or flow control valves 68, 72 and orifices C₁ or C₂ directly reduces the pressure in the respective chamber. For analysis, the above pressure expressions are linearized:

$${P}_{1} = \frac{{P}_{10} - \frac{β}{{V}_{1}} {A}_{p} r \dot{θ} - {K}_{c 1} {C}_{1}}{s + {K}_{p 1}} \\ {P}_{2} = \frac{{P}_{20} + \frac{β}{{V}_{2}} {A}_{p} r \dot{θ} - {K}_{c 2} {C}_{2}}{s + {K}_{p 2}} \qquad ( 4 )$$

For steady state conditions (\dot{θ}=0), the maximum pressure drop can be obtained through the continuity equation (assuming only one valve orifice, C₁, is open):

$$\begin{matrix}Q = {C}_{d} {C}_{0} \sqrt{\frac{2}{ρ} ( {P}_{s} - {P}_{1} )} \\ = {C}_{d} {C}_{1} \sqrt{\frac{2}{ρ} ( {P}_{1} )}\end{matrix} \qquad ( 5 )$$

Evaluating the expression, a closed form solution for the steady state chamber pressure is as follows:

$${P}_{1} = \frac{{( \frac{{C}_{0}}{{C}_{1}} )}^{2}}{1 + {( \frac{{C}_{0}}{{C}_{1}} )}^{2}} {P}_{s} \qquad ( 6 )$$

Therefore, if the variable orifice from the pressure relief valves 68, 72 is twice the area of the fixed orifice, the minimum chamber pressure is 0.2 Pₛ, Controlling C₀ directly controls the pressure through the above expression between Pₛ and 0.2 Pₛ. To provide symmetry on the torque, one valve can be controlled at a time. Therefore, C₁ controls negative torque and C₂ controls positive torque about axis 24.

The actuators 18 and 22 preferably have the same hydraulic characteristics, such as chamber dimensions, piston size and construction, ram size and construction, and orifice size and flow characteristics, in order to facilitate the balancing of actuator force delivered by each of the actuators 18 and 22 to the cam body 14. It is possible to provide the pressurized fluid to the actuators 18 and 22 from different supply lines, however, using a common supply line facilitates delivering the fluid at an equal pressure, and thereby the balancing of pressure in the cylinders and resulting force on the involute surfaces to zero the torque about the cam body 14.

An open fluid connection is provided between the hydraulic cylinders 42 and 46. The open fluid connection allows the hydraulic fluid to freely exit from one cylinder and enter another when the actuators 18 and 22 are in the balanced, no (or negligible) torque condition. As the cam body 14 is rotated and a piston in one of the cylinders is retracted, a volume of hydraulic fluid is expelled from that cylinder through the open fluid connection, and an equal volume of fluid enters the other cylinder. For a given angular movement of the cam body 14, the volume of fluid expelled from one cylinder will be the same as the volume of fluid entering the other. It is possible, however, to have different involute surfaces that are not mirror images, and differently dimensioned actuators, in which case for a given angular movement of the cam body 14 the movement of the pistons in each cylinder will be different, while maintaining the no torque condition. The open fluid connection can be a separate line, or can be the supply lines 54, 56 if they connected and constructed in such a way as to provide a direct pen fluid connection between the cylinders 42 and 46.

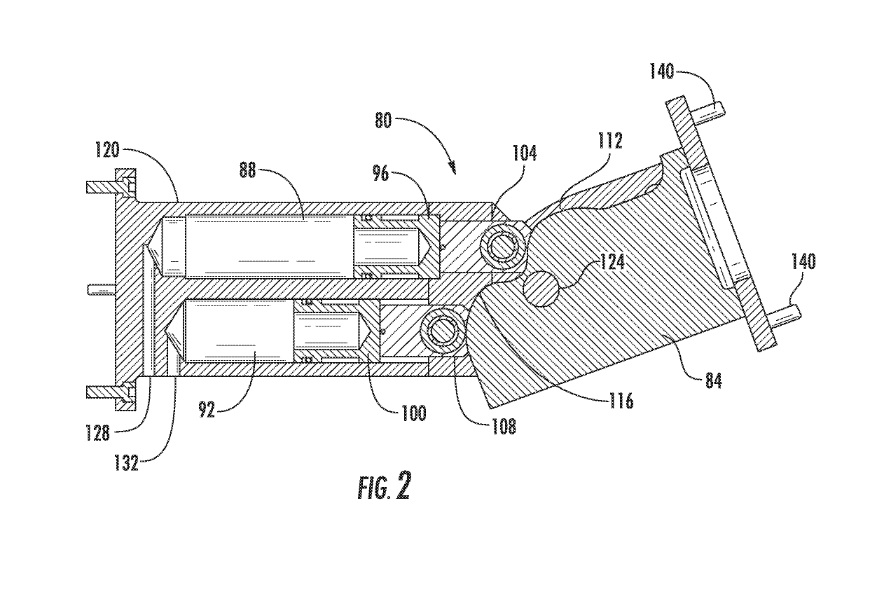

There is shown in FIG. 2 a hydraulic apparatus 80 having a cam body 84 and hydraulic actuators 88, 92. The hydraulic actuators 88, 92 have pistons 96, 100 that are connected to rams 104, 108. The rams 104, 108 are oriented so as to contact involute cam surfaces 112, 116 of cam body 84. The actuators 88, 92 can be provided in an actuator housing 120. The cam body 84 can be pivotally connected to the actuator housing 120 by pivot member 124 which also defines the axis of rotation of the cam body 84.

The actuators 88, 92 receive fluid through fixed fluid supply lines 128, 132 which can be connected to a common supply with pressure Pₛ. The release of fluid pressure from actuator 92 causes a differential in pressure between actuator 88 and actuator 92, which causes ram 104 of actuator 88 to extend against involute cam surface 112, while involute cam surface 116 exerts a retracting force on ram 108 of actuator 92. As the pivot point 124 and axis of rotation is between the two rams 104, 108 in this example, the result will be a net counterclockwise torque about the axis 124. Suitable fastening structure such as bolts 140 can be provided on the cam body 84 so that a tool can be attached to the cam body. Many different tools can be operated by a hydraulic apparatus according to the invention, including prosthetic tools.

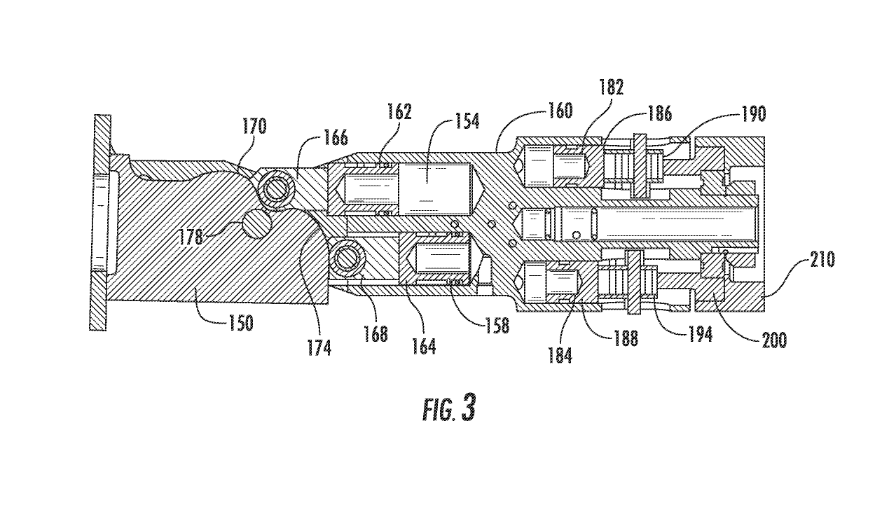

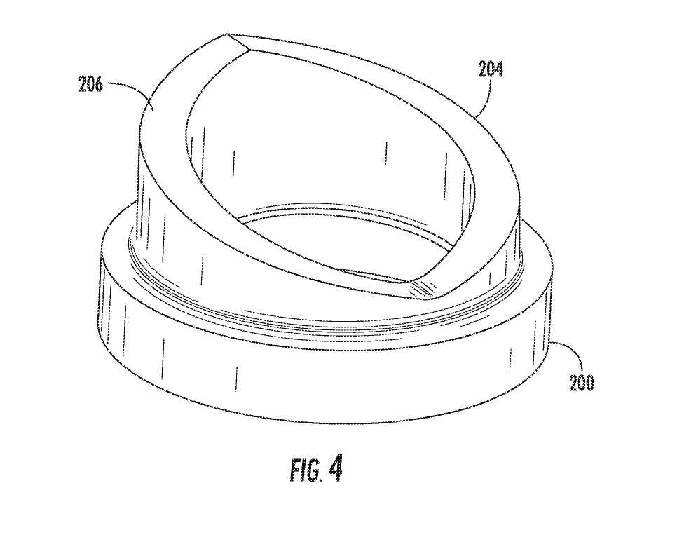

There is shown in FIG. 3 a hydraulic apparatus according to the invention in which a single involute cam is combined with a barrel cam. The barrel cam enables rotation about the axis of the link in combination with the transverse rotation achieved by the involute cam. The involute cam apparatus includes a cam body 150 and hydraulic actuators 154, 158, which can be provided in an actuator housing 160. The hydraulic actuators 154, 158 have pistons 162, 164 and rams 166, 168. The rams 166, 168 contact involute surfaces 170, 174 to cause the cam body 150 to rotate about the pivot 178. The barrel cam apparatus includes hydraulic actuators 182, 184 with pistons 186, 188 and rams 190, 194. The rams contact barrel cam 200 to impart rotation to the joint. The barrel cam 200 includes cooperating arcuate barrel cam surfaces 204, 206 (FIG. 4). Contact by the rams 190, 194 with the surfaces 204, 206 will cause rotation of the cam 200 and the barrel cam housing 210 to which it is connected.

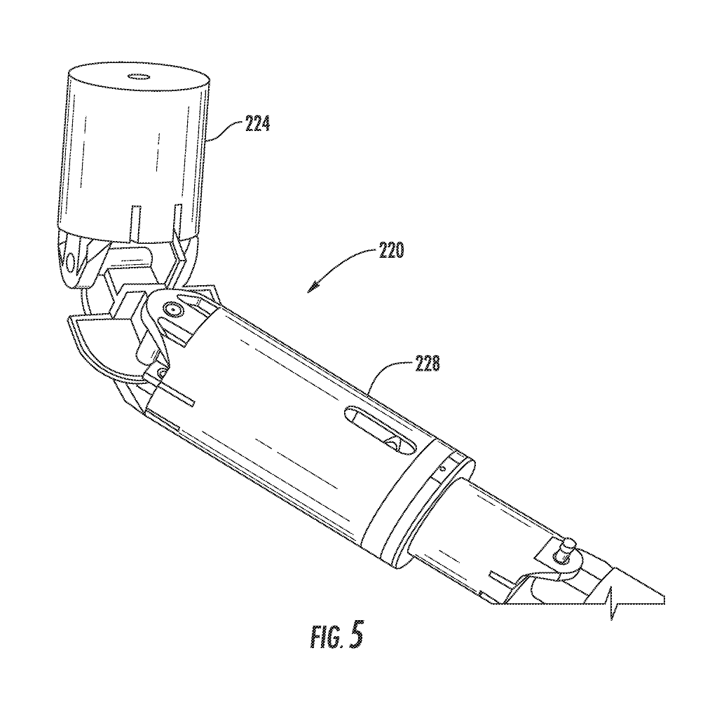

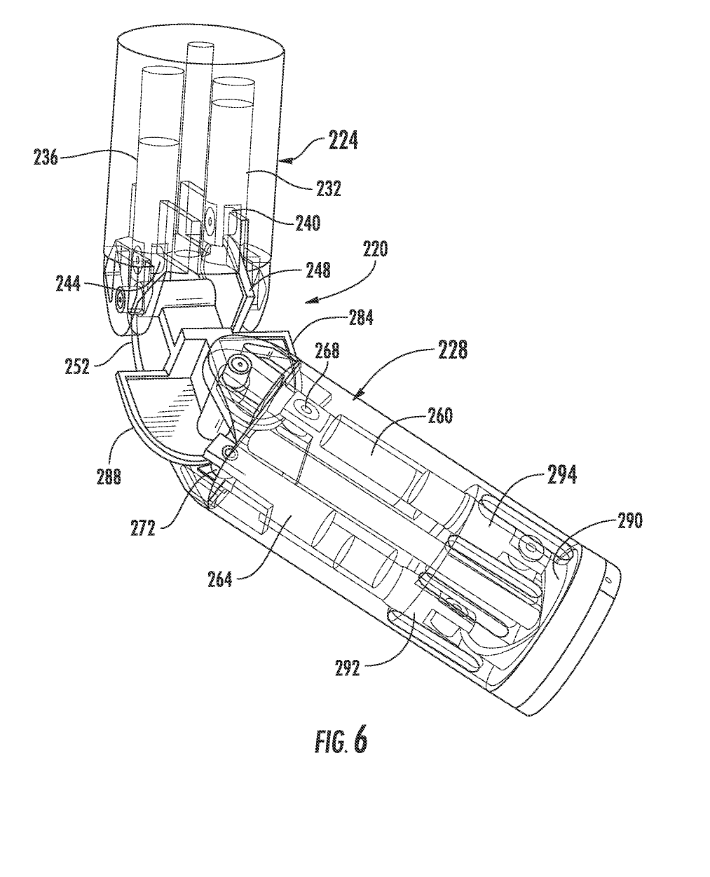

There is shown in FIGS. 5-7 a spherical joint according to the invention. The spherical joint provides for yaw, pitch and roll movement. The joint 220 includes first and second involute cam apparatus 224 and 228 which have involute cam surfaces that are orthogonal to one another. Hydraulic apparatus 224 comprises hydraulic actuators 232, 236 with pistons connected to rams 240, 244. The rams 240, 244 contact involute cam surfaces 248, 252 to impart yaw to the joint. Hydraulic apparatus 228 comprises hydraulic actuators 260, 264 with pistons connected to rams 268, 272. The rams 268, 272 contact involute cam surfaces 284, 288 to impart pitch to the joint. A barrel cam 290 and actuators 292, 294 can be provided to impart roll to the joint.

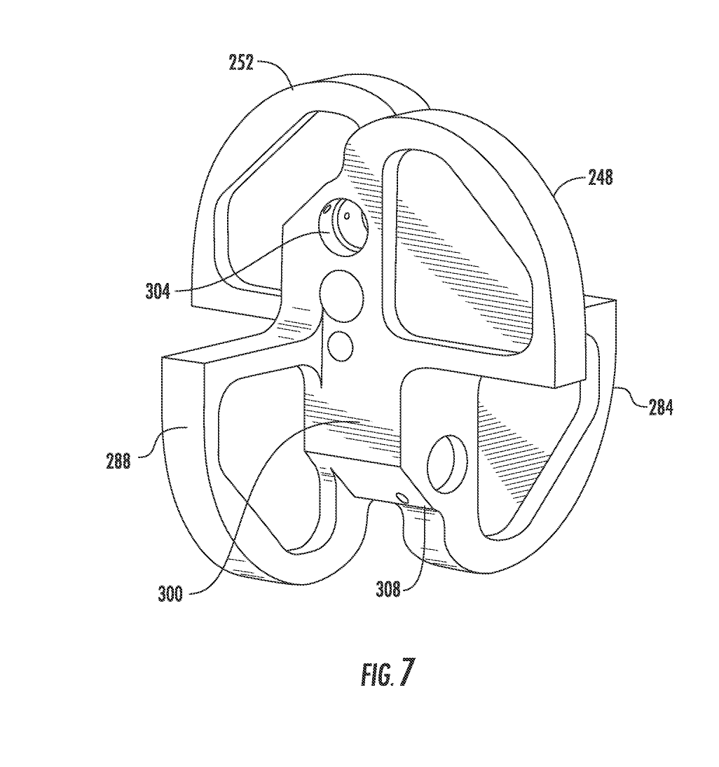

The involute cam surfaces 248, 252 of the hydraulic apparatus 224 and the involute cam surfaces 284, 288 of the hydraulic apparatus 228 can be provided on separate cam bodies. In the embodiment shown in FIG. 7 the involute cam surfaces 248, 252 and 284, 288 are provided on a single cam body 300. Orthogonal openings 304, 308 can be provided in the cam body 300 in which pivotal mountings can be positioned to permit the pivoting of the cam body 300 in both the yaw and pitch directions.

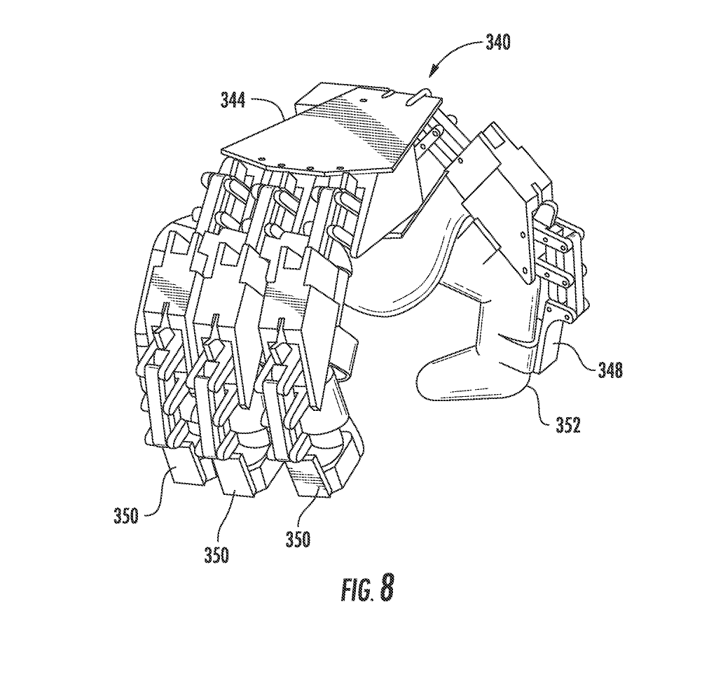

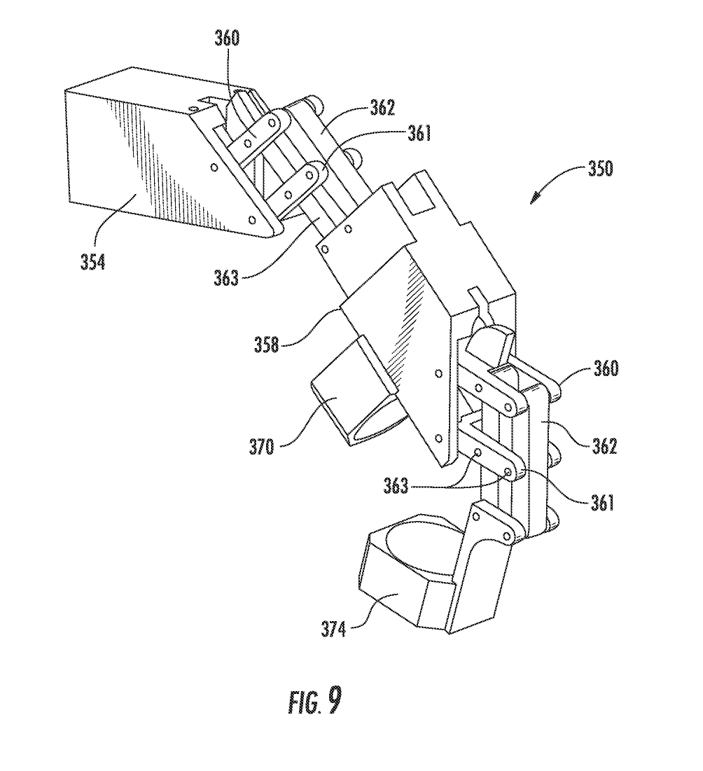

There is shown in FIGS. 8-10 an orthotic glove or hand 340 according to the invention. The hand 30 includes a palm member 344, a plurality of digits 350, and an opposing digit or thumb 348. The orthotic hand 340 can include a prosthesis 352 in the form of a hand, or the device can be used as a kind of wearable glove and attached to a users real hand to impart mobility to the hand.

The orthotic hand 340 can include a first hydraulic apparatus 354 and a second hydraulic apparatus 358. The first hydraulic apparatus 354 can be connected to the second hydraulic apparatus 358 by pivotal scissor connectors 360-363 to enable a remote center of rotation (enables joint rotation about the finger joint). Finger rings such as proximal ring 370 and distal ring 374 or other suitable engagement structure can be provided to engage the finger or orthosis in the area of the distal phalanges and the intermediate and/or proximal phalanges. Scissor connectors 360-363 can connect the second hydraulic apparatus 358 to distal ring 374.

Hydraulic apparatus 354 comprises hydraulic actuators 378, 382 and a cam body 386. The hydraulic actuators 378, 382 have pistons connected to rams 390, 394. The rams 390, 394 contact involute cam surfaces 398, 402 to impart rotation of the cam body 386 about pivot member 406. Cam body 386 is engaged to scissor connector 360, such that rotation of cam body 386 will pivot scissor connector 360, and through linkages will also move connectors 361-363.

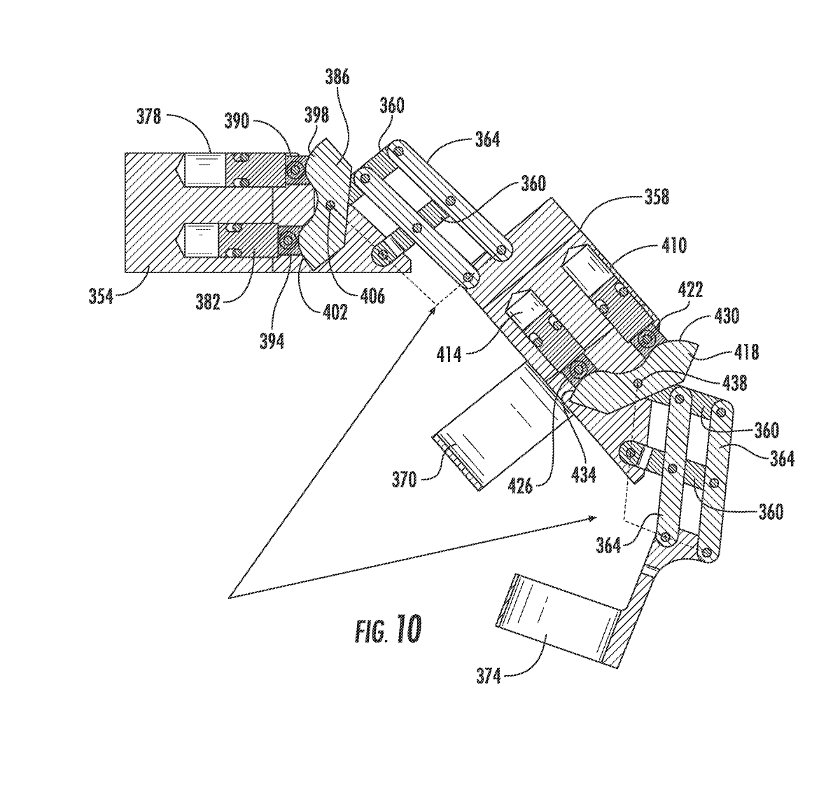

Hydraulic apparatus 358 comprises hydraulic actuators 410, 414 and a cam body 418. The hydraulic actuators 410, 414 have pistons connected to rams 422, 426. The rams 422, 426 contact involute cam surfaces 430, 434 to impart rotation of the cam body 418 about pivot member 438. Cam body 418 is engaged to associated scissor connector 360, and through linkages to connectors 361-363, such that rotation of cam body. The scissor mechanism enables joint rotation about the finger joint rather than the mechanism joint, as the point of rotation will be the intersection of the dashed lines in FIG. 10 as indicated by the solid arrows. This is important for mechanisms that are worn by the human body so that the mechanism axis of rotation (worn by the body coincident with the human joint axis of rotation.

In operation, the hydraulic actuators are selectively controlled by a suitable control device such as a computer, programmable controller, or other programmable device operating on pressure relief or flow control valves as previously described. Feedback devices such as proximity sensors may also be included to allow for accurate control throughout the range of motion. Joint position sensors provide pose information for the mechanism. Joint torque can be specified by computer models simulating virtual environments or from actual measured data from a remotely controlled robotic hand. Strain measurement or pressure measurement can be used to measure the actual joint torque for the controls. The selective operation of the actuators 378, 382 and associated rams 390, 394 for example will cause pivoting of the cam body 386, which will cause either extension or retraction of the scissor connectors 360, 364 and commensurate movement of the proximal ring 370 and associated part of the finger or prosthesis. Selective operation of the actuators 410, 414 and associated rams 422, 426 will cause pivoting of the cam body 418, which will cause either extension or retraction of the scissor connectors 360, 364 and distal finger ring 374 and associated part of the finger or prosthesis.

The invention is useful for many different kinds of orthosis, such as a hand, a shoulder, an arm, an elbow, a finger, a foot, an ankle, a knee, a hip, and a leg. The invention also can be used with robots, tools and machines that are not associated with or related to orthotics. Other component designs, shapes, dimensions, and the like are possible. Many different materials and hydraulic fluids can be utilized.

We claim:

1. A hydraulic apparatus with direct torque control comprising;
a cam body, the cat body being rotatable about an axis and having at least two mirror image involute cam surfaces on opposing sides of the axis;
at least two hydraulic actuators on opposing sides of the axis, each of the actuators having a linearly extendable ram and a hydraulic cylinder, the ram comprising a piston positioned in the hydraulic cylinder and each ram being extendable to contact the involute cam surface on the respective opposing side of the axis;
a fluid supply line for delivering a pressurized fluid to each of the cylinders;
an open fluid connection between the hydraulic cylinders;
a pressure relief or flow control valve associated with each of the hydraulic cylinders, the pressure relief or flow control valves being operable to selectively assume a closed position to retain fluid in the hydraulic cylinders or assume an open position to release fluid from the hydraulic cylinders; and
a control for operating the pressure relief or flow control valves between at least the closed and open positions, to extend and retract the rams out of and in to the cylinders to rotate the cam body about the axis with controlled torque.

2. The hydraulic apparatus of claim 1, further comprising a fixed area fluid supply orifice associated with each hydraulic cylinder, the supply orifices having an equal area and being connected to a common supply of pressurized fluid.

3. The hydraulic apparatus of claim 1, wherein the cylinders are provided in a cylinder housing, and the cam body is pivotally connected to the cylinder housing through the axis.

4. The hydraulic apparatus of claim 1, further comprising a tool connected to the cam body, whereby movement of the cam body will cause movement of the tool.

5. The hydraulic apparatus of claim 4, wherein the tool is a robotic, prosthetic or orthotic device.

6. The hydraulic apparatus of claim 5, wherein the robotic, prosthetic or orthotic device is selected from the group consisting of a hand, a shoulder, an arm, an elbow, a finger, a foot, an ankle, a knee, a hip, and a leg.

7. The hydraulic apparatus of claim 1, further comprising a barrel cam and cam follower, said barrel cam and cam follower imparting rotation about an axis orthogonal to the axis of rotation of the cam body.

8. The hydraulic apparatus of claim 1, wherein said cam body comprises two coincident pairs of involute cam surfaces.

9. The hydraulic apparatus of claim 1, further comprising a second hydraulic apparatus, said second hydraulic apparatus being connected in series to the other hydraulic apparatus.

10. The robotic, prosthetic or orthotic device of claim 1, wherein the valves are poppet valves.

11. A robotic, prosthetic or orthotic device including a hydraulic device, said hydraulic device comprising:
a cam body, the cam body being rotatable about an axis and having at least two mirror image involute cam surfaces on opposing sides of the axis;
at least two hydraulic actuators on opposing sides of the axis, each of the actuators having a linearly extendable ram and a hydraulic cylinder, the ram comprising a piston positioned in the hydraulic cylinder and being extendable to contact the involute cam surface on the respective opposing side of the axis;
a fluid supply line for delivering a pressurized fluid to each of the cylinders;
an open fluid connection between the hydraulic cylinders;
a pressure relief valve associated with each of the hydraulic cylinders, the pressure relief valves being operable to selectively assume a closed position to retain fluid in the hydraulic cylinders or assume an open position to release fluid from the hydraulic cylinders; and
a control for operating the pressure relief valves between at least the closed and open positions, to extend and retract the rams out of and in to the cylinders to rotate the cam body about the axis with controlled torque.

12. The robotic, prosthetic or orthotic device of claim 11, wherein a robotic, prosthetic or orthotic tool is connected to said cam body.

13. The robotic, prosthetic or orthotic device of claim 12, wherein said robotic, prosthetic or orthotic tool is selected from the group consisting of a hand, a shoulder, an arm, an elbow, a finger, a foot, an ankle, a knee, a hip, and a leg.

14. The robotic, prosthetic or orthotic device of claim 11, comprising first and second hydraulic devices connected in series.

15. The robotic, prosthetic or orthotic device of claim 14, wherein said first hydraulic device is connected to a robotic, prosthetic or orthotic hand.

16. The robotic, prosthetic or orthotic device of claim 15, further comprising a plurality of pairs of first and second hydraulic device digits, wherein said first hydraulic devices are connected to the robotic, prosthetic or orthotic hand, and another pair of first and second hydraulic devices connected to the hand as an opposable thumb.

17. A method of applying torque to a body, comprising the steps of:
providing a hydraulic device, said hydraulic device comprising a cam body, the cam body being rotatable about an axis and having at least two mirror image involute cam surfaces on opposing sides of the axis;
at least two hydraulic actuators on opposing sides of the axis, each of the actuators having a linearly extendable ram and a hydraulic cylinder, the ram comprising a piston positioned in the hydraulic cylinder and the ram being extendable to contact the involute cam surface on the respective opposing side of the axis;
a fluid supply line for delivering a pressurized fluid to each of the cylinders;
an open fluid connection between the hydraulic cylinders;
a pressure relief or flow control valve associated with each of the hydraulic cylinders, the pressure relief or flow control valves being operable to selectively assume a closed position to retain fluid in the hydraulic cylinders or assume an open position to release fluid from the hydraulic cylinders; and
a control for operating the pressure relief or flow control valves between at least the closed and open positions, to extend and retract the rams out of and in to the cylinders to rotate the cam body about the axis with controlled torque;
applying fluid pressure equally to the hydraulic cylinders to provide no torque on the cam body about the axis;
selectively releasing pressure in at least one of the hydraulic cylinders by operating a respective pressure relief valve, to create a hydraulic pressure differential between the hydraulic cylinders, the pressure differential creating a torque on the cam body about the axis and a net force on the respective ram of the reduced pressure hydraulic cylinder, causing the ram to retract into the respective hydraulic cylinder.
